(19) United States
(12) Patent Application Publication
(10) Pub. No.: US 2016/0194267 A1
(43) Pub. Date: Jul. 7, 2016

(54) Process For The Production Of Carboxylic Acid Esters

(71) Applicant: Johnson Matthey Davy Technologies Limited, London (GB)

(72) Inventors: Adrian Francis Backes, London (GB); Gabriella Eszter Toth, London (GB); Michael Gavin John Williams, London (GB)

(73) Assignee: Johnson Matthey Davy Technologies Limited, London (GB)

(21) Appl. No.: 14/916,234

(22) PCT Filed: Sep. 10, 2014
(86) PCT No.: PCT/GB2014/052734

§ 371 Date: Mar. 3, 2016

(30) Foreign Application Priority Data
Sep. 12, 2013 (GB) 1316269.8

(51) Int. Cl. C07C 67/08; B01J 19/24

(52) U.S. Cl. CPC C07C 67/08; B01J 19/24; B01J 2219/24

(57) ABSTRACT

In a process and apparatus for the production of carboxylic acid esters, a liquid carboxylic acid stream is fed to an upper section of a reaction zone maintained under esterification conditions. An alcohol vapour stream is fed to a lower section of the reaction zone; allowing the carboxylic acid stream to pass in countercurrent to the alcohol stream to form a liquid product stream of product ester. A refiner stream is withdrawn from at or near the top of the reaction zone and includes unreacted alcohol, water and ether by-product. The refiner stream is passed to a refining zone and treated to reduce the water content thereof to form an ether-containing stream having a water content that is lower than that of the upper stream removed from the reaction zone. This ether-containing stream is recycled to the reaction zone.

The present invention relates to a process for the production of carboxylic acid esters. In an alternative arrangement it relates to apparatus for the production of carboxylic acid esters. More particularly the present invention relates to a process and apparatus for the production of fatty acid esters. Still more particularly, the present invention relates to the production of ethyl esters of fatty acids.

Esterification is a well-known equilibrium-limited reaction involving the reaction of a mono-, di- or polycarboxlic acid or, in suitable cases, an acid anhydride, with an alcohol. The alcohol may be a mono, di- or polyhydric alcohol.

A process for carrying out esterification in a column reactor having a plurality of esterification trays is described in U.S. Pat. No. 5,536,856 the contents of which are incorporated herein by reference. Each esterification tray has a predetermined liquid hold-up and contains a charge of a solid esterification catalyst thereon. Examples of suitable catalysts include an ion exchange resin containing —SO₃H and/or —COOH groups. A liquid phase containing the carboxylic acid component, such as a fatty acid mixture, flows down the column reactor from one esterification tray to the next one against an upflowing alcohol vapour stream. The alcohol vapour is preferably methanol. Relatively dry alcohol vapour is injected into the bottom of the column reactor. Water of esterification is removed from the top of the column reactor in the vapour stream and ester product is recovered from the sump of the reactor. As the liquid flows down the trays it encounters progressively drier alcohol and the esterification equilibrium is driven further and further toward the desired 100% ester formation.

Whilst this process is very effective, particularly for the formation of methyl esters, if the desired ester is, for example, ethyl ester, economic problems are encountered in obtaining the relatively dry ethanol required for the process. This is because ethanol forms an azeotrope with water and is difficult to separate therefrom. Azeotropic ethanol will generally comprise about 5 weight % water and about 95 weight % ethanol and is known as “wet” ethanol. More particularly, “wet” ethanol may be 95.63 wt % ethanol and 4.37 wt % water. Suitable processes to achieve the dry ethanol required for the above process include material separation agent addition, pressure swing distillation and the use of molecular sieves. However, the processes available to obtain the required dry ethanol are expensive and therefore the use of dry ethanol impacts on the operating costs of the esterification reaction. If wet azeotropic ethanol were to be used in the above process, it is not sufficiently dry to enable complete, or near-complete, conversion of fatty acid to fatty acid ethyl ester to be achieved in prior art arrangements due to equilibrium constraints.

Interest in the production of fatty acid ethyl esters has increased as it has been suggested that fatty acid ethyl esters are a better biofuel in terms of performance and physical characteristics than the present commercial biofuels formed from fatty acid methyl esters. It is therefore desirable to provide a process which enables the wet azeotropic ethanol to be effectively and economically used in the production of the ethyl esters of fatty acids. Since the separation of alcohol from water is also problematic with other alcohols, the desired process will also offer advantages where the alcohol is other than ethanol.

In addition, as ethanol can be sourced from a sustainable feedstock via fermentation, it provides a more environmentally friendly approach to the production of fuels than methanol which is generally sourced from fossil fuels such as from natural gas or from coal gasification.

However, very high conversions, typically of the order of 99.7 to 99.8% of fatty acid, are required to meet the typical biodiesel specification of 0.5 acid value. This can be extremely difficult to achieve due to the esterification being an equilibrium reaction.

One process for producing organic acid esters is described in US2006/0014977. In this process the reaction is carried out using continuous countercurrent reactive distillation using acid catalysts in a structured packing in a single column. In one arrangement, absolute ethanol or an ethanol/water mixture is fed near the bottom of the column and a lactic acid solution in water is fed near to the top of the column. The ethyl lactate product is removed at the bottom of the reactor. In one arrangement, the ethanol-water azeotropic mixture is recycled back to the reaction distillation column. Whilst this process does appear to allow the use of wet ethanol, it does not produce a product which has the conversions required for biodiesel production.

An alternative process for esterifying a carboxylic acid to produce an ester using a wet alcohol vapour stream, in particular wet ethanol, is described in WO 2014/045034 the contents of which are incorporated herein by reference. The process includes the steps of: feeding a liquid carboxylic acid stream to an upper section of a first reaction zone maintained under esterification conditions; feeding a wet alcohol vapour stream comprising from about 3 to about 8 weight % water to a lower section of the first reaction zone; allowing the carboxylic acid stream to pass in countercurrent to the wet alcohol stream to form an intermediate liquid product stream comprising product ester and unreacted carboxylic acid; passing the intermediate liquid product stream to an upper section of a second reaction zone maintained under esterification conditions; feeding a dry alcohol stream to a lower section of the second reaction zone; allowing the intermediate product to pass in countercurrent to the dry alcohol stream such that further carboxylic acid is reacted to product ester; recovering the product ester stream; withdrawing a first stream comprising unreacted alcohol and water from the first reaction zone, treating the stream to reduce the water content to form a wet alcohol stream and feeding said stream to the first reaction zone; and optionally withdrawing a second stream comprising unreacted alcohol and water from the second reaction zone and supplying said stream to the first reaction zone.

Whilst this process is very effective and offers significant improvements over prior art arrangements, the conversion rate of carboxylic acid to product ester may be reduced where the reaction is carried out in an environment where ether by-product, for example diethyl ether, is produced. This is because for each mole of ether produced a mole of water is also produced and this causes a shift in the position of esterification equilibrium back towards higher carboxylic acid concentrations.

Where ethanol, or a higher alcohol, is used as the esterification alcohol, higher temperatures and liquid concentrations of alcohol are required in the base of the esterification column than are required where methanol is the alcohol in order to have a bubble point operating pressure. This is required to enable the alcohol vapour stream to pass up the column through the reaction stages. It will be understood that the operating pressure is also at a maximum in the base of the esterification column. These higher temperatures and concentrations result in higher dialkyl ether make. This problem is exacerbated as ether make tends to be significantly higher at the low acid, low water, concentrations found toward the base of the column than at the higher acid and water concentrations found toward the top of the reactor. This is particularly noted where ethanol is the selected alcohol and diethyl ester is the desired product.

However the desired dialkyl ester is produced, where the quality of the product stream from the esterification does not meet the specification requirements for biodiesel then further treatment will be required. This generally takes place in a polishing reactor. The presence of the polishing reactor downstream from the esterification column is disadvantageous as it increases both the capital and running costs of the process and will increase the total amount of alcohol required for the process.

There is therefore a need for a process which allows for a high conversion rate of carboxylic acid to product ester to be achieved when an ether by-product is produced. There is particularly a need for the process when “wet” alcohol is used. It has surprisingly been found that introducing a recycle of dialkyl ether into the base of the esterification column enables the required conversion to the desired ester to be achieved.

Thus, according to the present invention there is provided a process for the production of carboxylic acid esters by reaction of a carboxylic acid component and an alcohol component, said process comprising:
(a) feeding a liquid carboxylic acid stream to an upper section of a reaction zone maintained under esterification conditions;
(b) feeding an alcohol vapour stream to a lower section of the reaction zone;
(c) allowing the carboxylic acid stream to pass in countercurrent to the alcohol stream to form a liquid product stream comprising product ester and ether by-product;
(d) withdrawing a refiner stream from at or near the top of the reaction zone comprising unreacted alcohol; water and ether by-product;
(e) passing the refiner stream to a refining zone and treating said stream to reduce the water content thereof to form an ether-containing stream having a water content that is lower than that of the refiner stream removed from the reaction zone; and
(f) recycling the ether-containing stream from step (e) to the reaction zone.

By recycling the reduced water content ether by-product stream, a higher conversion rate of the carboxylic acid to the product ester is achieved than is achievable with prior art processes. One advantage of the present invention is that the desired conversion rate of carboxylic acid to product ester can be achieved in the presence of an ether by-product, without the need for separate processing of the product ester such as in a polishing reactor.

Without wishing to be bound by any theory it is believed that the introduction of the reduced water content ether stream into the lower section of the esterification zone alters the vapour-liquid equilibrium dynamics in the base of the reaction zone in a manner which is favourable to the desired reaction. For example, where the alcohol is ethanol, the diethyl ether (NBP=34.6° C.) is significantly more volatile than ethanol (NBP=78.3° C.) and this allows the base of the column to operate with significantly lower temperatures and alcohol concentrations than has been achievable heretofore. This in turn significantly reduces the diethyl ether formation rate on the lower trays such that the desired conversion can be achieved.

As diethyl ether is more volatile than ethanol and water, its separation from these components to form a reduced water content diethyl ether stream for recycling to the reaction zone is much easier than the separation of ethanol from water. Similar advantages are achieved with higher alcohols.

A further advantage is that the recycled ether can be regarded as an inert which facilitates three phase mixing and aids in the removal of the water of esterification. Since the recycled ether is a by-product of the reaction the requirement to add an external inert such as nitrogen, which is required in prior art systems, may be avoided.

Although a dialkyl ether, such as diethyl ether, is much more volatile than water and the alkanol from which the dialkyl ether is formed, and its separation from them can be regarded as relatively straightforward, it does form an azeotrope with water, albeit at a relatively low concentration of about 1.25 weight %. This complicates the separation of a lower water content. However, the inventors of the present invention have surprisingly found that the addition of a small amount of dry make-up alcohol to the refining zone helps reduce the water content of the dialkyl ether to form the reduced water content dialkyl ether stream, as the water preferentially associates with the alcohol. Thus according to one arrangement of the present invention, dry alcohol is added to the refining zone.

In one arrangement, the conversion rate of carboxylic acid to product ester is at least about 99.0%, at least about 99.5%, at least about 99.6%, at least about 99.7%, or at least about 99.8%. Consequently, the product ester may have an acid value of 0.5 mg KOH/g or less and therefore meet the requirements for biodiesel.

The esterification reaction may be carried out in any suitable reaction zone. In one arrangement it may be a single reactor. In one arrangement, the reaction zone will comprise a plurality of esterification trays. Although two or three trays may suffice in some cases, it will typically be necessary to provide at least about 5 to about 40 or more esterification trays in the reaction zone.

Typically each esterification tray is designed to provide a residence time for liquid on each tray of from about 1 minute up to about 120 minutes, preferably from about 5 minutes to about 60 minutes.

In one arrangement, the esterification reaction in the reaction zone may be carried out in the presence of a catalyst. Any suitable catalyst may be used. Suitable catalysts include acidic ion exchange resins containing —SO₃H and/or —COON groups. Macroreticular resins of this type may be useful. Examples of suitable resins are those sold under the trade marks ‘Amberlyst’, ‘Dowex’, ‘Dow’ and ‘Purolite such as Amberlyst 13, Amberlyst 66, Dow C351 and Purolite C150.

Where a catalyst is used in the reaction zone, different catalysts may be used on different trays. Moreover, different concentrations of catalyst may be used on different trays.

The charge of catalyst on each tray, where present, is typically sufficient to provide a catalyst:liquid ratio on that tray corresponding to a resin concentration of at least 0.2% w/v, for example a resin concentration in the range of from about 2% w/v to about 20% w/v, preferably 5% w/v to 10% w/v, calculated as dry resin. Sufficient catalyst should be used to enable equilibrium or near equilibrium conditions to be established on the tray within the selected residence time at the relevant operating conditions. However, the amount of catalyst used on each tray should not be so large that it becomes difficult to maintain the catalyst in suspension in the liquid on the tray by the agitation produced by the upflowing vapour entering the tray from below. For a typical resin catalyst a resin concentration in the range of from about 2% v/v to about 20% v/v, preferably 5% v/v to 10% v/v may be used.

The particle size of the catalyst should be large enough to facilitate retention of the catalyst on each tray by means of a screen or similar device. However, as the larger the catalyst particle size is the more difficult it is to maintain it in suspension and the lower the geometrical surface area per gram, it is expedient to use not too large a catalyst particle size. A suitable catalyst particle size is in the range of from about 0.1 mm to about 5 mm.

In one arrangement, a treatment bed may be located above any reaction zone which includes the catalyst to remove potential resin poisons such as metals which may be present in the carboxylic acid component. Such poisons may be present where natural acids or acids from, for example, cooking oils are used.

One or more wash trays may be provided above the esterification trays in order to prevent loss of product, solvent and/or reagents from the reaction zone.

The reduced water content ether stream may be recycled to any suitable place in the reaction zone. Generally, where the reaction zone is a single reactor it will be recycled to the lower section of the reaction zone.

In one alternative arrangement, the reaction zone may be split into a first reaction zone and a second reaction zone, each having respective upper and lower sections. The first and second reaction zones may be located in separate reaction vessels or may be separate zones within a single reaction vessel. For convenience, the first and second reaction zones are ordered in the direction of flow of the acid feed.

Where the reaction zone comprises first and second reaction zones, the refiner stream removed for treatment of the ether may be withdrawn from an upper section of the first reaction zone and the reduced water content ether stream to be recycled to the reaction zone may be fed to a lower section of the second reaction zone and optionally to a lower section of the first reaction zone.

Where first and second reaction zones are present, the split of trays between the first and second reaction zones may be the same or different. In one arrangement, the first reaction zone may have more trays than the second reaction zone. In particular, the first zone may have about 10 trays and the second zone may have about 5 trays.

Where first and second reaction zones are present, the carboxylic acid component, preferably in the form of a liquid carboxylic acid stream, may be fed to an upper section of the first reaction zone. The liquid carboxylic acid stream may be supplied as high as possible within the first reaction zone to maximise contact with the alcohol component.

The alcohol component, which may be in the form of a wet alcohol vapour stream, may be fed to a lower section of the first reaction zone. The carboxylic acid component may be allowed to pass countercurrent to the alcohol component in the first reaction zone to produce an intermediate product stream, preferably in the liquid phase, comprising product ester and unreacted carboxylic acid.

In this arrangement, the intermediate product stream may be fed to an upper section of the second reaction zone. The intermediate product stream may require cooling before it is fed to the second reaction zone. The requirement for cooling will generally be dictated by the requirement to minimise ether formation in the second reaction zone, whilst at the same time maintaining suitable reaction conditions. For example, the stream may be cooled from a temperature of about 110° C. to about 200° C. to a temperature of from about 70° C. to about 130° C. Make-up alcohol, preferably in the form of a dry alcohol vapour stream, may be fed to the second reaction zone, preferably to a lower section thereof. The intermediate product stream may be allowed to pass countercurrent to the alcohol component in the second reaction zone such that further carboxylic acid is reacted to product ester. A product ester stream may be recovered from the second reaction zone.

In this arrangement, the majority of the esterification will generally take place in the first reaction zone. Preferably, around 90% to 95% conversion of the carboxylic acid is performed in the first reaction zone with the remainder being carried out in the second reaction zone.

Optionally in an arrangement where there is a first and second reaction zone, a second stream comprising ether by-product, unreacted alcohol and water may pass from the second reaction zone to the first reaction zone. Where this is the case, it is advantageous that the second reaction zone is located directly below the first reaction zone such that the second stream simply flows upwardly into the first reaction zone.

Where first and second reaction zones are present, the reaction in the first reaction zone may be carried out in the absence of a separate catalyst. That is to say it may be autocatalysed. Alternatively, at least a portion of the first reaction zone may be free of catalyst. This catalyst free area in the first reaction zone will generally be located towards the top of the reaction zone.

Whichever arrangement is used for the reaction zone(s), the refiner stream comprising unreacted alcohol, ether by-product and water is removed from the reaction zone and passed to a refining zone where it is treated to reduce the water content. The alcohol in this stream will generally have a relatively high water content. The reaction zone and the refining zone may be located in separate vessels or may be separate zones within a single vessel.

In the arrangement where the reaction zone comprises a first and second reaction zone, the refiner stream may be removed from an upper section of the second reaction zone and passed to the refining zone. In this arrangement, the first reaction zone may only receive alcohol recycled from the refining section and not be fed directly with vapour from the second reaction zone.

The unreacted alcohol may be treated in the refining zone. Where the refining zone comprises an alcohol refiner, either alone or in combination with an ether refiner, the alcohol is treated in the alcohol refiner to reduce the water content to form a wet alcohol stream which may be recycled to a lower section of the first reaction zone.

Where the refining zone comprises an alcohol refiner, the ether by-product may have its water content reduced in a pasteurisation section located towards the top of the alcohol refiner. In one arrangement the alcohol refiner will be a distillation column. The distillation column may be operated at any suitable conditions. In one arrangement it may have a pressure of from about 0.1 bara to about 15 bara. In one alternative, the pressure is from about 1.0 bar to about 3.0 bars. The temperature of the column may be from about 5° C. to about 200° C. In one alternative, the temperature is from about 35° C. to about 120° C. The pressure in the ether pasteurisation portion of the refining zone may be from about 0.1 bara to about 15 bara. In one arrangement, the pressure may be from about 1.0 bara to about 1.6 bara. The temperature of the column in this section may be from about 35° C. to about 160° C. In one arrangement it may be from about 45° C. to about 120° C.

In an alternate arrangement, the ether refining column may be separate from the alcohol refining column. In this arrangement, a stream from the top of the alcohol refining column containing alcohol, water, and ether is passed to an ether refining column. Ether having a water content lower than the water content of the stream passed to the ether refining column is removed from at or near the top of the ether refining column and recycled to the esterification reaction.

Whether the alcohol and ether refining columns are separate or combined in a single column, dry ethanol make-up may be supplied at, or near, the top of the ether refining column to assist in the separation of the ether from the water.

In an alternative arrangement, the refining zone may comprise an alcohol refiner in combination with a packed bed to assist in water removal from the ether stream. In one arrangement, the packed bed may be packed with molecular sieves. In this arrangement, the unreacted alcohol and any ether may be treated in the alcohol refiner to reduce the water content to form a wet alcohol which is then passed to the molecular sieve apparatus to form a dry alcohol and ether stream. This treatment may happen simultaneously with the ether by-product treatment such that the resultant reduced water content ether stream is a combined dry alcohol/ether stream. The dry alcohol/ether stream may be fed to a lower section of both the first and second reaction zones or to a lower section of the reaction zone.

In one arrangement, where the ether and the alcohol are recycled together, a wet alcohol make-up may be supplied to the packed bed together with a wet ether and alcohol recycle from the refining zone, and a combined ether and alcohol stream having a lower water content returned to the reaction zone. This may be particularly important if the packing is a molecular sieve.

Since the ether, such as diethyl ether, is more volatile than the alcohol, such as ethanol, and water, its separation from these components to form a dry diethyl ether stream for recycling to the reaction zone is relatively straightforward.

The ether returned to the reaction zone will have a lower water content than that removed from the reaction zone in the stream removed from the top of the reaction column. In one arrangement it may have a water content of about 0.01 weight % to about 1 weight %, preferably about 0.01 weight % to about 0.2 weight %.

The carboxylic acid component may be supplied to the reaction zone as a carboxylic acid stream. Preferably, the carboxylic acid component is in the liquid phase.

The process of the present invention may be for the production of a monoester. Examples of monoesterification reactions include the production of alkyl esters of aliphatic mono-carboxylic acids from alcohols and aliphatic monocarboxylic acids. Any suitable monocarboxylic acid may be used but in one arrangement, it may contain from about 6 to about 26 carbon atoms and may include mixtures of two or more thereof. Examples of monocarboxylic acids include fatty acids such as decanoic acid, dodecanoic acid, tetradecanoic acid, hexadecanoic,acid, octadecanoic acid, octadecenoic acid, linoleic acid, eicosanoic acid, isostearic acid and the like, as well as mixtures of two or more thereof.

Mixtures of fatty acids are produced commercially by hydrolysis of naturally occurring triglycerides of vegetable origin, such as coconut oil, rape seed oil, and palm oils, and triglycerides of animal origin, such as lard, tallow and fish oils. If desired, such mixtures of acids can be subjected to distillation to remove lower boiling acids having a lower boiling point than a chosen temperature (e.g. C₈ to C₁₀ acids) and thus produce a “topped” mixture of acids, or to remove higher boiling acids having a boiling point higher than a second chosen temperature (e.g. C₂₂₊acids) and thus produce a “tailed” mixture of acids, or to remove both lower and higher boiling acids and thus produce a “topped and tailed” mixture of acids. The resultant mixture may be the carboxylic acid component supplied to the reaction zone.

The fatty acid mixtures may contain ethylenically unsaturated acids such as oleic acid.

In one alternative, the process of the present invention may be used to carry out a diesterification. Thus the process can be used to produce dialkyl esters of aliphatic and cycloaliphatic C₄ to C₁₈ saturated and unsaturated dicarboxylic acids. These can be produced by reaction of alcohols with the dicarboxylic acids or anhydrides thereof, or with mixtures of the dicarboxylic acid and its anhydride. Dialkyl oxalates, dialkyl maleates, dialkyl succinates, dialkyl fumarates, dialkyl glutarates, dialkyl pimelates, and dialkyl azelaates are examples of dicarboxylic acid esters which may be produced by the process of the present invention. Another example of a suitable carboxylic acid is tetrahydrophthalic acid. The C₁ to C₁₀ alkyl esters of these dicarboxylic acids are of particular interest. Either the free dicarboxylic acid or its anhydride (if such exists) or a mixture of dicarboxylic acids and anhydride can be used as the carboxylic acid component starting material for production of such dialkyl esters.

Alkyl esters of aromatic C₇ to C₂₀ monocarboxylic acids and mixtures thereof can be made by a process of the invention. Benzoic acid and 1-naphthoic acid are examples of such acids.

Alkyl esters of aromatic C₈ to C₂₀ dicarboxylic acids can also be produced by the process of the invention from the acids, their anhydrides and mixtures thereof.

It is also possible to produce polyalkyl esters of polycarboxylic acids by the process of the invention. Suitable polycarboxylic acid moieties include, for example, citric acid, pyromellitic dianhydride, and the like.

Carboxylic acid esters of dihydric and polyhydric alcohols can be produced by the process of the invention. Examples of these esters include ethylene glycol diformate, ethylene glycol diacetate, propylene glycol diformate, propylene glycol diacetate, glyceryl triacetate, hexose acetates, and the acetate, propionate and n-butyrate esters of sorbitol, mannitol and xylitol, and the like.

It will be understood that the carboxylic acid stream supplied to the reaction zone may be a stream comprising a mixture of carboxylic acids.

The alcohol component may be supplied to the reaction zone as an alcohol stream. Preferably, the alcohol component is a vapour under the conditions in the reaction zone.

Suitable alcohols include those having from 1 to 10 carbon atoms. However, generally the process of the present invention is not generally of economic benefit where methanol is the alcohol since di-methyl ether has a low boiling point of −24° C. and so it is difficult to condense at moderate pressures such that chiller units may be required. In one arrangement the alcohol has from 2 to 5 carbon atoms, for example, ethanol, propanol, isopropanol, butanol and pentanol. Methanol and ethanol are particularly preferred as the alcohol used in the present invention. In one arrangement, a mixture of alcohols may be used. The mixture may in one arrangement be a mixture of methanol and ethanol or a mixture of methanol, ethanol and propanol and/or isopropanol.

The alcohol may be provided as a wet alcohol stream or as a conventional dry alcohol. By ‘wet’ alcohol we mean alcohol having an azeotropic amount of water or more and by ‘dry’ alcohol we mean alcohol having a water content that is less than azeotropic. More specifically, where the alcohol is ethanol, the wet alcohol may comprise about 4.4 wt % water or more and the dry alcohol may comprise less than about 4.4 weight % water. Generally, where it is desirable to produce a product which is suitable for use in biodiesel, a dry alcohol stream will be used.

The reaction conditions required in the reaction zone will depend on the carboxylic acid component and alcohol component selected for the reaction. In one arrangement, where first and second reaction zones are present and particularly where the alcohol component is ethanol, the first reaction zone may require temperatures in the range of from about 90° C. to 160° C. and preferably in the range of from about 120° C. to about 130° C. The second reaction zone may require temperatures in the range of from about 70° C. to about 150° C. and preferably in the range of from about 90° C. to about 120° C.

Typical operating pressures within the reaction zone are from about 0.1 bar to about 20 bar and preferably from about 1.8 bar to about 3.4 bar. Where first and second reaction zones are present, the first reaction zone is preferably operated at a lower pressure than the second reaction zone.

According to another aspect of the present invention there is provided apparatus for use in a process for the production of carboxylic acid esters by reaction of a carboxylic acid component and an alcohol component, said apparatus comprising:
a reaction zone comprising an upper inlet for the introduction of a liquid carboxylic acid feed, a lower inlet for the introduction of a make-up alcohol, a lower outlet for the withdrawal of product ester, an upper outlet for the withdrawal of a refiner stream comprising an ether by-product and water, and an inlet for the introduction of reduced water content ether from the refining zone, said reaction zone being configured to operate under esterification conditions;
conduit to transport the refiner stream from the reaction zone to a refining zone;
the refining zone comprising an inlet for receiving the upper stream from the reaction zone, an outlet for removing reduced water content ether, said refining zone being configured to operate such that the ether removed at the outlet has a lower water content then the ether fed to the inlet; and
conduit to return the reduced water content ether to the inlet for the introduction of reduced water content ether to the reaction zone.

In a preferred arrangement, the reaction zone will additionally include an inlet for the introduction of reduced water content alcohol and the refining zone will additionally include an outlet for removing reduced water content alcohol, and the refining zone is configured to operate such that the alcohol removed at the outlet has a lower water content than the feed to the inlet; and the apparatus will additionally include conduit to recycle the reduced water content alcohol stream to the inlet to the reaction zone.

Any suitable arrangement for the reaction zone may be used. Examples of suitable arrangements are discussed above in connection with the process of the present invention. In particular, the reaction zone may comprise a first and second reaction zone.

Any suitable arrangement for the refining zone may be used. Examples of suitable arrangements are discussed above in connection with the process of the present invention. In particular, the reaction zone may comprise a first and second reaction zone.

The present invention will now be described by way of example with reference to the accompanying figures in which:

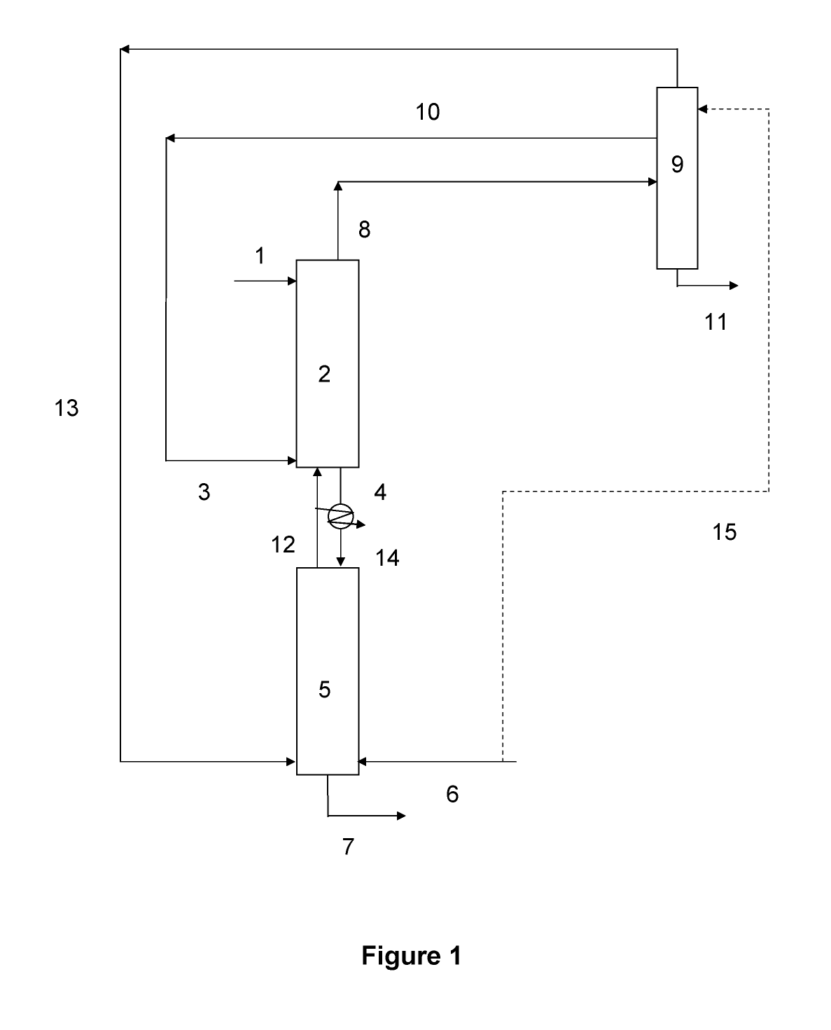

FIG. 1 is a schematic representation of a process according to a first aspect of the present invention;

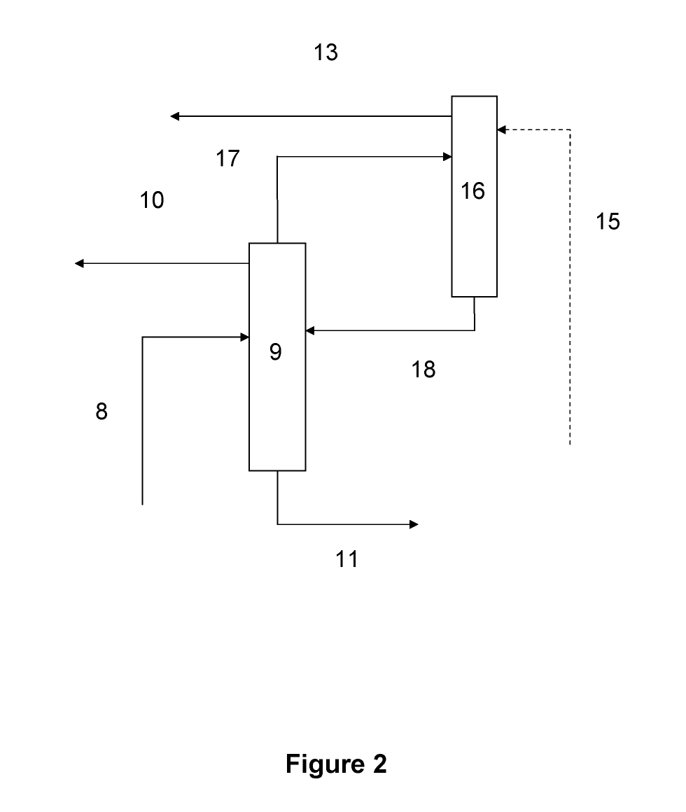

FIG. 2 is a schematic representation of a process according to a second aspect of the present invention;

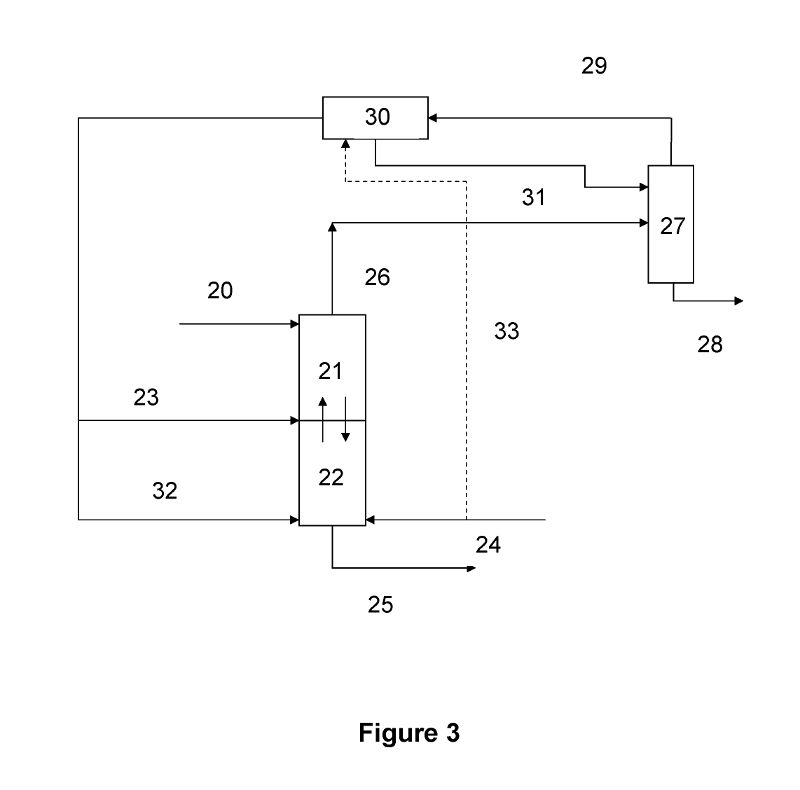

FIG. 3 is a schematic representation of the process according to a third aspect of the present invention; and

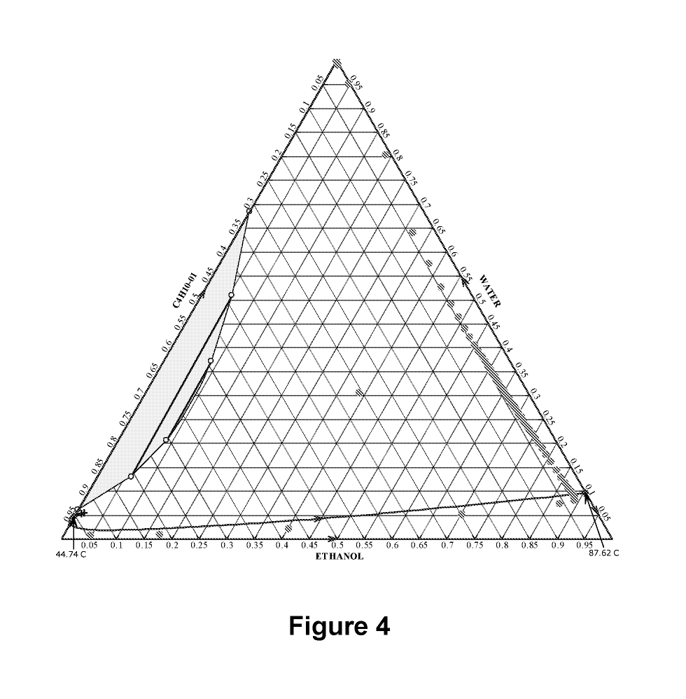

FIG. 4 is a ternary diagram depicting the molar composition profiles of Example 1

It will be understood by those skilled in the art that the drawings are diagrammatic and that further items of equipment such as reflux drums, pumps, vacuum pumps, temperature sensors, pressure sensors, pressure relief valves, control valves, flow controllers, level controllers, holding tanks, storage tanks, and the like may be required in a commercial plant. The provision of such ancillary items of equipment forms no part of the present invention and is in accordance with conventional chemical engineering practice.

One arrangement of the present invention is illustrated schematically in FIG. 1. A stream comprising carboxylic acid is added in line 1 to the first reaction zone 2. This flows downwardly and contacts an increasingly dry alcohol vapour stream. The alcohol, which may be wet alcohol, is added into the first reaction zone in line 3. As the wet alcohol stream travels upwardly it reacts with the downflowing carboxylic acid to form product ester together with ether by-product and water. The wet alcohol stream and ether by-product travel upwardly, becoming wetter as a result of the water produced in the esterification and etherification reactions.

The vapour containing the water of reaction, the water in the initial alcohol stream, unreacted alcohol and ether by-product is removed in line 8. This stream is fed to an alcohol refiner 9 such as a distillation column where excess water is separated from the alcohol and ether by-product and is removed in line 11. In one arrangement, dry alcohol make-up stream may be provided to the alcohol refiner 9 in line 15 to assist the separation of water from the ether. Wet alcohol from the refiner 9 is returned in line 10 and hence to line 3 from where it is fed to the first reaction zone 2.

An ether stream having a lower water content than the ether feed to the refiner 9 in line 8 is removed from the top of the refiner 9 and fed to the bottom of the second reaction zone 5 in line 13.

A liquid stream comprising an intermediate product stream comprising ester product and unreacted carboxylic acid is removed from the base of the first reaction zone 2 and fed in line 4 to the second reaction zone 5 via cooler 14. The stream flows downwardly through the second reaction zone 5 encountering progressively drier vaporous alcohol and ether. Dry make-up alcohol is added in line 6. Product ester is removed from the reactor in line 7.

Unreacted alcohol together with the water of esterification and etherification and ether recycle and by-product is removed from the top of the second reaction zone in line 12 and passed to the bottom of the first reaction zone 2. This stream will comprise wet alcohol.

Whilst this process has been illustrated in connection with a two stage reaction zone and with the use of wet alcohol it will be readily understood that the arrangement of the refiner 9 can be applied to a conventional single reaction zone system or to a multi-zone reaction system.

A modification of part of the process illustrated in FIG. 1 is set out in FIG. 2. Components of the system which are the same as in FIG. 1 have the same reference numerals and their function is as described in connection with FIG. 1.

In one arrangement, the ethanol refiner 9 of FIG. 1 is replaced with a dual refining column arrangement which may be nominally described as an alcohol refining column and an ether refining column. In this arrangement, the vapour removed from the first reaction zone in line 8 is fed to the alcohol refiner 9 where excess water is separated from the alcohol and the ether by-product and is removed in line 11. A wet alcohol stream is returned to the first reaction zone via line 10.

The ether by-product in the stream flows upwardly to the top of the alcohol refiner 9 where it is fed to a separate ether refiner 16 in line 17. Further excess water is separated from the ether in the ether refiner 16. Some alcohol may also be separated in the refiner. This excess water and optionally alcohol is returned from the bottom of the ether refiner 16 to the alcohol refiner 9 in line 18. An ether stream having a lower water content than the stream fed to the ether refiner 16 is fed to the bottom of the second reaction zone in line 13. A dry alcohol make-up stream is provided to the ether refiner in line 15.

A still further arrangement is illustrated in FIG. 3. In this arrangement, the first 21 and second 22 reaction zones are combined in a single vessel. A stream comprising carboxylic acid is added in line 20 to the first reaction zone 21. This stream flows downwardly contacting an increasingly dry alcohol/ether vapour stream which is added to the reactor in line 23. The partly reacted carboxylic acid stream continues downwardly into the second reaction zone 22 where it contacts a dry alcohol/ether vapour stream added in line 32 and dry make-up alcohol added in line 24. The product ester is removed in line 25. If all of the ether and alcohol is added via line 32, then the first and second reaction zone are effectively combined into a single reaction zone.

As the dry alcohol/ether flows upwardly through the second reaction zone it gathers the water of esterification and etherification so that by the time it reaches the first reaction zone 21 it is a wet alcohol/ether stream. The wet stream travels up through the first reaction zone 21 where it picks up additional water of esterification and etherification. This stream is recovered in line 26 and is sent to an alcohol refiner 27, where excess water is separated from the alcohol and ether components and removed in line 28. The remaining stream, a wet stream of alcohol and ether, will be removed from the top of the alcohol refiner 27 and fed to molecular sieve apparatus 30 in line 29.

In the molecular sieve apparatus 30, further excess water is separated from the alcohol and ether components to form a stream of dry alcohol/ether with very low water content i.e. below azeotropic levels. This stream of dry alcohol/ether will be fed to the first reaction zone 21 in line 23 and the second reaction zone 22 in line 32. The excess water and some ether and alcohol passes from the molecular sieve apparatus 30 to the alcohol refiner 27 in line 31 where the ether and alcohol is further refined before being fed back to the molecular sieve apparatus 30 and the water is removed in line 28. Optionally, all of the make-up alcohol may be sent to the molecular sieve apparatus 30 in line 33 if its water content is not sufficiently low.

The present invention will now be described with reference to the following example. Whilst the example is specifically directed to the recycling of diethyl ether and ethanol, it will be understood that the method described is equally applicable to other alcohols and ether by-products.

### EXAMPLE 1

A vapour stream (Stream 1) containing ethanol, diethyl ether and water was drawn from a first reaction zone and fed to an ethanol refiner. The ethanol refiner was a 58-stage ethanol distillation column operating at 1.45 bara overhead. The stream was fed into the ethanol refiner 6 stages from the base of the column. As the stream progressed up the column, excess water was separated from the ethanol and diethyl ether.

A wet ethanol stream (Stream 2) in the liquid phase was drawn from the ethanol refiner 45 stages from the base of the column and was fed to the base of the first reaction zone. An excess water stream (Stream 3) was drawn from the base of the ethanol refiner and disposed of.

A dry diethyl ether stream (Stream 4) in the liquid phase was drawn from the top of the ethanol refiner and fed to the base of a second reaction zone.

For each of Streams 1 to 4, the mass flow rate, mole fractions, mass fractions, total flow rate and temperature were recorded. The data is presented in Table 1 below.

TABLE 1

| | Stream | | | |
|---|---|---|---|---|
| | 1 | 2 | 3 | 4 |
| Mass Flow (kg/h) | | | | |
| Ethanol | 967 | 928 | 0 | 38 |
| Water | 295 | 63 | 231 | 2 |
| Diethyl ether | 1,215 | 52 | 0 | 1,145 |
| Mole Fractions | | | | |
| Ethanol | 0.390 | 0.829 | 0.000 | 0.050 |
| Water | 0.305 | 0.143 | 1.000 | 0.007 |
| Diethyl ether | 0.305 | 0.029 | 0.000 | 0.942 |
| Mass Fractions | | | | |
| Ethanol | 0.390 | 0.891 | 0.000 | 0.032 |
| Water | 0.119 | 0.060 | 0.999 | 0.002 |
| Diethyl ether | 0.491 | 0.049 | 0.000 | 0.966 |
| Total Flow (kg/h) | 2,477 | 1,042 | 231 | 1,185 |
| Temperature (° C.) | 108 | 83 | 125 | 46 |

From the results it can be seen that treatment in the ethanol refiner successfully separates the water from the diethyl ether by-product to form a dry diethyl ether stream (Stream 4) with a 0.007 mole fraction and 0.002 mass fraction of water. The dry diethyl ether stream can be fed into the reaction zone without reducing the conversion rate of carboxylic acid to product ester.

FIG. 4 illustrates a ternary plot by composition in 58 stage ethanol column with diethyl ether pasteurisation on molar basis.

1. A process for the production of carboxylic acid esters by reaction of a carboxylic acid component and an alcohol component, said process comprising:
(a) feeding a liquid carboxylic acid stream to an upper section of a reaction zone maintained under esterification conditions;
(b) feeding an alcohol vapour stream to a lower section of the reaction zone;
(c) allowing the carboxylic acid stream to pass in countercurrent to the alcohol stream to form a liquid product stream comprising product ester;
(d) withdrawing a refiner stream from at or near the top of the reaction zone comprising unreacted alcohol; water and ether by-product;
(e) passing the refiner stream to a refining zone and treating said stream to reduce the water content thereof to form an ether-containing stream having a water content that is lower than that of the upper stream removed from the reaction zone; and
(f) recycling the ether-containing stream from step (e) to the reaction zone.

2. The process according to claim 1 wherein dry alcohol is added to the refining zone.

3. The process according to claim 1 wherein the reaction zone is one of a single reaction zone and two reaction zones comprising a first and second reaction zone wherein the first reaction zone is located above the second reaction zone.

4. The process according to claim 3 wherein the reduced water content ether stream is recycled to a lower section of a single reaction zone.

5. The process according to claim 3 wherein the reduced water content ether stream is recycled to a lower section of a first reaction zone in a two reaction zone arrangement.

6. The process according to claim 1 wherein the refining zone comprises an ether refiner one of alone and in combination with an alcohol refiner.

7. The process according to claim 6 wherein alcohol is treated in the alcohol refiner to reduce the water content to form a wet alcohol stream which is recycled to the reaction zone.

8. The process according to claim 6 wherein the water content of the ether is reduced in a pasteurisation section located towards the top of the alcohol refiner.

9. The process according to claim 6 wherein the ether refining column is separate from the alcohol refining column.

10. The process according to claim 6 wherein dry ethanol make-up is supplied to the ether refining column.

11. The process according to claim 1 wherein the refining zone comprises an alcohol refiner in combination with a packed bed for water removal from the ether.

12. The process according to claim 11 wherein the packed bed is packed with molecular sieves.

13. The process according to claim 11 wherein a wet alcohol make-up is supplied to the packed bed for water removal.

14. The process according to claim 1 wherein the alcohol is ethanol.

15. Apparatus for use in a process for the production of carboxylic acid esters by reaction of a carboxylic acid component and an alcohol component, said apparatus comprising:
a reaction zone comprising an upper inlet for the introduction of a liquid carboxylic acid feed, a lower inlet for the introduction of an alcohol, a lower outlet for the withdrawal of product ester, an outlet for the withdrawal of a refining stream comprising an ether by-product and water, and an inlet for the introduction of reduced water content ether from the refining zone, said reaction zone being configured to operate under esterification conditions;
a conduit to transport the upper stream from the reaction zone to a refining zone;
the refining zone comprising an inlet for receiving the refining stream from the reaction zone, an outlet for removing reduced water content ether, said refining zone being configured to operate such that the ether removed at the outlet has a lower water content then the ether fed to the inlet; and
a conduit to return the reduced water content ether to the inlet for the introduction of reduced water content ether to the reaction zone.

16. Apparatus according to claim 15 wherein the reaction zone further comprises an inlet for the introduction of reduced water content alcohol and the refining zone further comprises an outlet for removing reduced water content alcohol, the refining zone being configured to operate such that the alcohol removed at the outlet has a lower water content than the alcohol feed to the inlet, the apparatus further comprising a conduit to recycle the reduced water content alcohol stream to the inlet to the reaction zone.
